(19) United States
(12) Patent Application Publication
(10) Pub. No.: US 2009/0281537 A1
(43) Pub. Date: Nov. 12, 2009

(54) METHOD AND APPARATUS FOR TREATING A FUNGAL NAIL INFECTION WITH SHORTWAVE AND/OR MICROWAVE RADIATION

(75) Inventors: Alexander Britva, Migdal Haemek (IL); Sharon Feldman, Ramat Hasharon (IL); Ziv Karni, Kfar Shmaryahu (IL); Joseph Lepselter, Nordiya (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Alma Lasers Ltd., Caesarea (IL)

(21) Appl. No.: 12/441,930

(22) PCT Filed: Sep. 20, 2007
(86) PCT No.: PCT/IL07/01156

§ 371 Date: Mar. 22, 2009

(30) Foreign Application Priority Data
Sep. 21, 2006 (US) 11533776

(51) Int. Cl. A61B 18/18

(52) U.S. Cl. 606/33

(57) ABSTRACT

The present invention relates to methods and devices for treating hard biological tissue (in particular, keratin-rich hard tissues) with electromagnetic energy having a frequency of at least 0.5 MHZ (megahertz) and less than 10 GHZ (gigahertz) (for example, HF, RF or microwave energy), and particularly, to methods and devices for treating infections, for example, fungal infections of the nail.

### FIELD OF THE INVENTION

The present invention relates to methods and devices for treating hard biological tissue (in particular, keratin-rich hard tissues) with electromagnetic energy having a frequency of at least 0.5 MHZ (megahertz) and less than 10 GHZ (gigahertz) (for example, HF, RF or microwave energy), and particularly, to methods and devices for treating infections, for example, fungal infections of the nail.

### BACKGROUND OF THE INVENTION

### Nail Fungus

Fungal infections of the nail, referred to by the terms “nail fungus,” “onychomycosis,” or “tinea unguium,” are common throughout the world. An estimated 2-13% of the population is affected in North America, with at least 15-20% of those aged 40-60 having one or more fingernails or toenails infected. Toenails are much more commonly affected than fingernails. Infections can range from superficial, causing little more than discoloration, to severe, resulting in loss of the nail together with deformities of the surrounding digit. The incidence of onychomycosis has been rising over the past few decades, due to factors such as an increased elderly population, increased participation in vigorous physical activity while wearing moisture-retaining shoes and socks, an increase in the number of HIV infected individuals, an increased incidence of diabetes, and increased use of steroids, antibiotics, and other therapeutics that can suppress immunologic responses to fungi.

While nail fungus is rarely life threatening, it causes significant pain, inconvenience, embarrassment, emotional distress, and limitations to manual performance and ambulation. Individuals with moderate to severe onychomycosis can lose their ability to perform many routine tasks (such as fastening buttons, picking up small objects, walking significant distances) and can lose the ability to perform satisfactorily in their occupations. Due to the unpleasant appearance of their hands or feet, these individuals may become socially self-conscious and embarrassed, and may avoid intimate or other close contact with people. Loss of self-esteem, anxiety, and depression commonly result from moderate to severe cases of fungal nail infection.

At present, topical treatments for nail fungus are rarely effective. Although some oral antifungal therapies have moderate efficacy, they also pose significant risks of toxic reactions, and many patients would prefer local treatments to systemic treatments.

### Etiology and Varieties

Onychomycosis is caused most commonly by several species of dermatophytes (parasitic fungi that infect the keratin-rich tissue of the stratum corneum, hair, and nails) and, less commonly, by nondermatophyte molds or by yeasts, primarily of the genus Candida. An estimated 90% of cases are caused by dermatophytes, primarily of the genera Trichophyton, Microsporum, and Epidermophyton, while about 8% are caused by nondermatophyte molds and 2% by yeasts. The causative agent in onychomycosis can rarely be determined by clinical appearance; microscopic examination or culturing is usually required. Furthermore, an infected nail colonized by one species can develop secondary infections by other fungi, yeasts, or bacteria.

Onychomycosis can affect the nail plate, the nail bed (the tissue directly under the nail plate), the nail matrix or nail root (the thickened skin at the base of the nail from which the nail plate develops), the cuticle, the hyponychium (the thickened layer of epidermis beneath the free end of the nail), and the proximal and lateral nail folds (the skin adjacent to the base and sides of the nail).

Factors that contribute to the development of onychomycosis include advanced age, diabetes (which reduces circulation to the extremities), wearing heat- and moisture-retaining footwear, communal bathing, HIV infection, the use of antibiotics or immunosuppressive drugs, trauma to the nail, use of insufficiently cleaned manicure tools, poor overall health, and warm climates.

Onychomycosis can be categorized into several varieties based on clinical appearance. The recognized varieties include:

Distal and lateral subungual onychomycosis (DLSO): This is the most common variety. It usually results from a fungal infection of the skin (usually the plantar skin of the foot) spreading to the nail bed and then to the underside of the nail plate via the hyponychium. The distal and lateral parts of the nail plate become thickened, white, and opaque. In addition, the nail bed becomes hyperkeratotic and onycholysis (separation of the nail plate from the bed, ultimately resulting in loss of the nail) commonly ensues. Paronychia (inflammation of the tissues adjacent to the nail) is also common. Trichophyton rubrum is the most common pathogen.

Endonyx onychomycosis (EO): This is a variety of DLSO in which the fungus spreads directly from the skin to the nail plate rather than to the nail bed. The nail again is thickened, white, and opaque, but there is no evident nail bed hyperkeratosis or onycholysis.

White superficial onychomycosis (WSO): This variety is almost always found on toenails. The surface of an infected nail develops white dots or powdery patches and the nail becomes rough and crumbly. Trichophyton mentagrophytes (T. interdigitale) is the most common cause, though some nondermatophyte molds such as Acremonium, Aspergillus, and Fusarium can also infect the upper surface of the nail plate. The nondermatophyte molds commonly cause black or green nails.

Proximal subungual onychomycosis (PSO): In this least common variety, the fungus first attacks the cuticle and proximal nail fold, and then penetrates the proximal nail plate. The distal part of the nail remains normal.

Candida onychomycosis (CO): The yeast, nearly always Candida albicans, infects the nail folds (paronychia), the nail plate and surrounding tissues (in chronic mucocutaneous candidiasis), the nail bed, or any combination of these. The entire digit commonly becomes swollen and deformed. Candida may cause onycholysis or it may colonize onycholytic nails (resulting from trauma or another infection). Candida infection associated with paronychia is almost always secondary to trauma to the nail folds.

Total dystrophic onychomycosis (TDO): The entire nail plate is thickened, yellow-brown, and opaque. All the adjacent tissues are affected, and the nail matrix may be permanently damaged, preventing normal nail growth even after the infection resolves. TDO can be the endpoint of any of the other onychomycosis varieties.

A patient with onychomycosis may have one variety or any combination of varieties.

### Current Treatments

Onychomycosis is presently treated primarily with oral antifungal agents. Topical agents are rarely effective by themselves, except in mild cases that only affect the distal nail plate. They may, however, be beneficial in combination with oral therapy. In severe cases, the affected nail (and sometimes the nail bed and matrix) is removed surgically or by use of a urea containing formulation; removal of the nail is done in conjunction with oral and sometimes topical therapy. Further details on some of these methods follow.

Oral medications: The preferred therapy for onychomycosis is orally administered treatment with terbinafine (Lamisil™), itraconazole (Sporanox™), or fluconazole (Diflucan™). Terbinafine, an allylamine, is active against dermatophytes, but has considerably less efficacy against nondermatophyte molds and against yeasts. Itraconazole and fluconazole are triazoles that are effective against dermatophytes, nondermatophyte molds, and yeasts. When administered daily, all of these compounds can cause hepatic injury, and monitoring of liver enzymes is required. Pulse therapy (typically, administration one week per month) reduces the risks for hepatic damage, but prolongs the course of therapy from about 6 to 12 weeks to at least several months. Terbinafine has several potentially serious drug interactions, and the triazoles, because they are metabolized using the hepatic cytochrome P450 system, have numerous significant drug and food interactions that prevent their use in many patients. Even though these drugs are the currently preferred treatments for onychomycosis, their cure rates are not high: a recent survey (Arch Dermatol 134(12):1551-1554, 1998) found that standard treatment with terbinafine resulted in disease-free nails in approximately 35-50% of cases, while the rate for itraconazole was approximately 25-40%. Relapse is also common, though precise figures are not available. These oral therapies are nevertheless more effective than topical treatments because they apparently penetrate the nail more quickly and thoroughly, and because they remain in the nail for weeks or months following treatment.

Topical treatments: Topical antifungal treatments are now administered mainly in cases where the fungal infection is restricted to the distal half of the nail plate or in cases in which the patient cannot tolerate oral therapy. Again, their low efficacies appear due mainly to their inability to adequately penetrate the nail. Topical antifungal agents include allylamines (including terbinafine), triazoles (including itraconazole and fluconazole), imidazole derivatives (including ketoconazole, miconazole, clotrimazole, and enconazole), amorolfine, ciclopirox olamine, sodium pyrithione, bifonazole plus urea, and propylene glycol plus urea plus lactic acid.

Surgical Treatments: One problem that must be overcome when treating fungal infections of the nail is that the fungus spores are located deep under the thick nail. Thus, many physicians recommend trimming or removing some or all of the nail plate to allow topical medications to reach the locations in the nail bed where fungal spores are located. Unfortunately, removal of the nail can cause considerable discomfort to the patient, both at the time that the procedure is performed, and during the time period where the new nail has not grown in and replaced the removed nail.

Existing treatments for onychomycosis are thus of limited efficacy, have high risks for adverse effects and drug interactions, and are time consuming and inconvenient for the patient. Thus, there is an ongoing need for improved methods and apparatus for treating hard tissue (for example, the nail and adjacent tissue), for treating fungal infections, for in particular, for treating fungal infections of the nail.

Treatment of Skin with RF Radiation

There are a number of disclosures related to treatment of skin and underlying soft tissue with RF radiation. For example, US 2003/0220635 of Knwolton et al., entitled “Method for Treating Skin and Underlying Tissue,” teaches a method for treating a tissue site by coupling an energy delivery surface of an RF energy delivery device with a skin surface. It is disclosed that a reverse thermal gradient is created by cooling the skin surface, and heat delivered from the RF energy device treats tissue beneath the skin surface. A number of applications of the method are disclosed, including treating wrinkles, inducing formation of scar collagen, reducing acne scars, injuring the sebaceous gland, reducing activity of bacteria that creates acne dermal tightening hair follicle modification, spider vein removal, modification of fat tissue, and modification of muscle tissue. There is no disclosure or suggestion of treating finger or toe nails, no disclosure or suggestion of coupling an energy-delivery surface with a hard surface (for example, a nail surface), and no disclosure or suggestion of using RF energy to treat fungal infections.

PCT IL05/000314, entitled “Improved System and Method for Heating Biological Tissue Via RF Energy,” of the present inventors, teaches a method and device for treating a selected target within a subject (for example, biological tissue) by configuring an RF energy source, a phase shifter, an impedance matching network, and an RF resonator to deliver a traveling wave of RF energy. According to examples presented, energy is delivered via a rigid (for example, metal) applicator which serves as an RF-energy coupler. According to these examples, upon contact between the rigid applicator and the epidermis of the patient, the skin conforms to a shape of the applicator to provide a necessary “tight contact” between an applicator surface and the epidermis.

It is disclosed that device and method are effective for RF (HF) energy treatment of deep layers of human tissue (e.g. dermis and/or hypodermis) to achieve adipose tissue contraction and/or cellulite reduction.

There are several mechanisms associated with RF-heating of biological tissues. For example, rotational movement of dipolar water molecules in the alternating electromagnetic fields and the corresponding electromagnetic wave, and tissue resistance to conductive current have both been cited as mechanisms. It is believed that the last mechanism is especially important. Thus, when using RF energy for skin tightening, it is observed that heating is primarily in adipose tissue because it is rich in liquids but not subject to convective cooling as blood vessels are.

To date, there is no teaching or suggestion of using high frequency energy (e.g. shortwave/RF or microwave) to treat tissue that typically has a relatively low water concentration. To date, there is no teaching or suggestion of using high frequency energy to treat fungal nail infection.

### SUMMARY

The present inventors are now disclosing for the first time that, surprisingly, administration of ‘high frequency’ radiation (e.g. HF, RF and/or microwave radiation) is useful for treating infected fingernails and/or toenails, despite the fact that human nails are known to typically have a low water content (i.e. 10-15%).

Towards this end, the present inventors are disclosing an electromagnetic energy delivery device that is operative, in exemplary embodiments, upon delivery of ‘high-frequency’ electromagnetic energy (i.e. electromagnetic energy having a frequency of at least 0.5 MHZ (megahertz) and less than 10 GHZ (gigahertz)) through a surface of the nail plate, to create a sharp temperature gradient in the nail plate and/or nail bed. Thus, in exemplary embodiments, the thermal/temperature gradient is generated such that the nail plate and/or connective tissue between the nail bed and the nail plate and/or “upper layers” of nail bed tissue (i.e. a portion of or the entire nail bed epidermis and optionally at least a portion of the nail bed dermis) are heated to a temperature sufficient for reducing fungus pathogen activity and/or reducing a population of fungus (for example, between 50 degrees and 80 degrees, or between 60 degrees and 70 degrees), while deeper-lying tissue are not heated, or heated to a lesser extent. Thus, the presently disclosed temperature gradient may be useful for targeting fungus pathogens residing in the region where the nail meets the skin with minimal or no damage to lower-lying tissue.

In exemplary embodiments, the high frequency electromagnetic energy (e.g. HF, RF and/or microwave energy) is delivered via an energy delivery surface of a conformable conducting applicator, including but not limited to a soft or flexible applicator. Upon contact (for example, application of pressure) between the applicator and the fingernail and/or toenail, a surface of the applicator conforms and/or yields to a shape of an upper surface of the nail to provide a “tight contact” between the opposing surfaces. Thus, in particular embodiments, the presently-disclosed conformable conductive applicator serves as an electromagnetic energy coupler that functions, in combination with the upper surface of the nail place (for example, via the “tight contact” between the relatively hard nail plate and the softer conformable applicator), as a lossy transmission line (where losses are determined by biological tissue absorption of the high-frequency-energy) for delivering electromagnetic energy (e.g. HF, RF and/or microwave energy) to the nail plate and/or tissue beneath the nail plate (for example, the nail bed or upper layers thereof).

Not wishing to be bound by theory, it is noted that there is variation between the sizes and shapes of different human nails. As such, in order for the applicator surface to provide a “tight contact” with nails of different size and shape, it may be advantageous to use an applicator with a surface that “conforms” to the human nail surface, for example, a soft applicator that conforms to the shape of the hard upper surface of the human nail. Appropriate materials from which the applicator may be constructed include but are not limited to rubber, elastometric material, knitted materials, string, strips, etc. If the applicator is constructed primarily from a material not sufficiently conductive for the applicator to function as an electrode (for example, rubber, which is constructed from insulating rubber), it is possible to associate a second more conducting material with the insulating conformable (for example) so material. For example, conducting pieces of material (for example, metal) may be embedded in a conformable, soft, less conducting matrix.

It is noted that because human nails typically have a relative low water content, it may be useful to pre-treat the nail (for example, with a chemical agent such as a urea-containing formulation) to increase the water concentration of the nail plate. Not wishing to be bound by theory, it is believed that the additional water molecules within the nail plate, when subjected to the high frequency alternating electromagnetic field, rotate to increase the temperature of the nail plate and/or adjacent tissue.

In exemplary embodiments, the energy is delivered as a traveling wave of electromagnetic (e.g. HF, RF and/or microwave) energy, and the device includes a mechanism (for example, including an impedance transformer) so that the traveling electromagnetic wave may pass through the upper surface of the nail plate substantially without being converted to a standing wave and dissipated later in the nail tissue.

As used herein, when the traveling electromagnetic wave passes through a surface substantially without being converted to a standing wave, this means that power of a reflected electromagnetic wave (if any) is low (less than 10%) and usually lower than 1-2%.

As noted earlier, in exemplary embodiments, the delivered electromagnetic energy is localized to specific locations below the surface of the upper surface of the nail plate so that the “localized” energy only does not penetrate too deeply below the surface of the nail bed which could burn the patient and/or inflict pain. This may be useful, for example, for reducing collateral damage, a risk of burning, coagulating and/or pain to the patient. This localization of heating may be accomplished using one or more techniques.

Thus, in some embodiments, a mechanism for phase controlling a location of maximum amplitude of the traveling electromagnetic energy wave is provided. Thus, the electromagnetic energy device may include, in some embodiments, a phase shifter capable of shifting a phase of directed traveling waves. In exemplary embodiments, the phase shifter is configured so that a location of a maximum of the traveling energy wave is at or near the surface of the nail plate in order to facilitate the heating process and/or sharpen an achievable temperature gradient.

In some embodiments, delivery of electromagnetic energy as a series of one or shorter, intense electromagnetic power pulses may also be useful for confining the heat to specific locations. Thus, in exemplary embodiments, the high-frequency radiation is delivered in a series of one or more RF-power pulses (for example, rectangular pulses) of short duration, for example, having pulse duration of between 10 nanoseconds and 10 milliseconds. In exemplary embodiments, the pulse duration is between 1 and 100 milliseconds. Towards this end, the presently disclosed energy device may include a pulse controller, operative to control pulse parameters so that the electromagnetic energy source delivers an output signal in pulses of predetermined duration and amplitude with a desired frequency. For example, the electromagnetic (e.g. HF, RF and/or microwave) energy may be produced in the form of a sinusoidal signal that can be modulated by rectangular pulses with a lower frequency by pulse width modulation.

According to another aspect of the present invention there is provided apparatus for treating a human nail (i.e. finger nail and/or toe nail) of a subject, the apparatus comprising:

a) an electromagnetic energy source operative to produce ‘high frequency’ electromagnetic energy having a frequency of at least 0.5 MHZ (megahertz) and less than 10 GHZ (gigahertz), the electromagnetic energy source configured to produce an output electromagnetic signal (i.e. a signal of the ‘high frequency’ electromagnetic energy having a frequency of at least 0.5 MHZ (megahertz) and less than 10 GHZ (gigahertz)) directed to a conductive conformable applicator (coupler) contactable with an upper surface of the nail of the subject;

b) a pulse controller operative to cause the electromagnetic energy source to deliver the output electromagnetic signal as a pulsed signal having one or more pre-determined pulse parameters, the pulse controller operative to effect a pulse-width modulation of the pulsed signal;

c) an impedance transformer operative to convert an impedance of tissue associated with the nail so that traveling waves of the pulsed output signal reach the human nail substantially without being converted to a standing wave; and

d) the conductive conformable applicator for delivering the electromagnetic pulsed output signal to the nail, the conformable applicator configured to conform to a shape of the upper surface of the nail plate upon contact with the nail, wherein the apparatus is operative such that the delivery of the pulsed signal is effective to heat at least one of a nail plate tissue and nail bed tissue below the nail plate.

In exemplary embodiments, the electromagnetic energy source selected from the group consisting of a HF energy source (i.e. operative to produce so-called HF radiation having a frequency between 0.5 kHz and 220 MHZ), an RF radiation source (i.e. operative to produce short wave radiation in the 2-100 MHz range) and a microwave radiation source (i.e. operative to produce microwave radiation having a frequency between 0.87 and 2.45 GHz).

According to some embodiments, the apparatus is operative to deliver the pulse signal to heat at least one of a nail plate tissue and nail bed tissue below the nail plate to a temperature sufficient to inhibit activity of a pathogen residing within the heated tissue (for example, for the case of fungal pathogens, at least 50 degrees Celsius, or at least 60 degrees Celsius).

According to some embodiments, the apparatus is operative to deliver the pulse signal to heat at least one of a nail plate tissue and nail bed tissue below the nail plate to a temperature sufficient to inhibit activity of a fungal pathogen residing within the heated tissue.

According to some embodiments, the apparatus is operative to deliver the pulse signal to heat at least one of a nail plate tissue and nail bed tissue below the nail plate to at least 50 degrees Celsius

According to some embodiments, the apparatus is operative such that the heating produces a temperature variation of at least 20 degrees/millimeter over a distance of at least 0.1 mm in at a location below the surface of the nail plate

According to some embodiments, the location of the temperature gradient is within the nail plate.

According to some embodiments, the location is within 1 mm of a nail plate-nail bed interface.

According to some embodiments, the location is within a given distance from the nail plate-nail bed that is at most the thickness of the nail plate.

According to some embodiments, the apparatus is operative to produce the sharp temperature gradient in accordance with the one or more pre-determined pulse parameters of one or more pulses delivered from the energy source.

According to some embodiments, at least one pulse parameter is selected from the group consisting of amplitude, pulse duration, a pulse shape, a duty cycle parameter, a pulse sequence parameter, a pulse rise-time, and a pulse frequency.

According to some embodiments, the pulse generator is operative to establish a value of the duty cycle parameter that is between 1% and 100%.

According to some embodiments, the pulse generator is operative to establish a value of the duty cycle parameter that is between 15% and 30%.

According to some embodiments, the pulse generator is operative to establish a value of the pulse duration between 10 nanoseconds and 10 milliseconds.

According to some embodiments, the pulse generator is operative to establish a rectangular pulse shape.

According to some embodiments, the apparatus further comprises: e) a phase shifter operative to control a phase of the traveling waves of the pulsed output signals so that a location of maximum amplitude is substantially at the upper surface of the nail plate within a tolerance that is at most 0.5 a thickness of the nail plate.

According to some embodiments, the tolerance is at most 0.2.

According to some embodiments, the conformable conductive applicator includes:
a) a first non-conducting conformable material; and b) a plurality of particles of a second conducting material, the particles embedded within the first material.

According to some embodiments, the first non-conducting material is an electrical insulator.

According to some embodiments, a ratio between a volume of the non-conducting material and an aggregate volume of the plurality of particles is at least 3.

According to some embodiments, conformable conductive applicator includes at least a flexible portion.

According to some embodiments, the conformable conductive applicator includes a dielectric coating.

According to some embodiments, the apparatus is operative to deliver a dose (for example, total quantity of energy, energy treatment duration, amplitude, duty cycle of energy delivery) of energy determined in accordance with a thickness of the nail plate.

According to some embodiments, the applicator is movable on the surface of the biological tissue as a means of altering an energy delivery region.

According to some embodiments, the apparatus has a VSWR of 1.05-1.2

According to some embodiments, the apparatus further includes e) a RF resonator located in the applicator, the resonator operative to cyclically accumulate and release a desired amount of energy to the nail via the applicator.

According to some embodiments, the apparatus further includes a feeding cable, the feeding cable connecting the conductive conformable applicator and the resonator with the impedance transform.

According to some embodiments, the feeding cable has a resonance length defined by n*l/2 length, where l is a wavelength of energy in the cable material and n is a whole number.

According to some embodiments, the impedance transformer (i.e. impendence matching network) includes a fixed structure characterized by a shape selected from the group consisting of L shaped, T shaped and P-shaped structure.

According to some embodiments, the IMN includes a broadband impedance transformer.
According to some embodiments, the IMN is variable.

According to some embodiments, the phase shifter includes a trombone type.

According to some embodiments, the phase shifter is at least partially constructed of coaxial cable.

According to some embodiments, a phase shift provided by the phase shifter is variable.

According to some embodiments, coupled energy is delivered from RF-generator (amplifier).

According to some embodiments, the energy delivered to the predetermined energy dissipation zone is coupled in continuous and/or pulsing mode.

According to some embodiments, the electromagnetic energy (e.g. RF and/or microwave and/or HF-energy) is characterized by a resonance frequency which matches a known natural resonance frequencies of the selected target.

According to some embodiments, the apparatus further comprises at least one additional component selected from the group consisting of a laser beam, an ultrasonic transducer, a UV light source, a plasma treatment device and a flash lamp.

According to some embodiments, the apparatus further comprises: e) a nail thickness measurer (for example, laser based), for measuring a thickness of the nail; f) an energy dose calculator for calculating an energy dosage in accordance with the measured thickness, wherein the apparatus is operative to provide energy in accordance with results of the calculating of the energy dose.

It is now disclosed for the first time apparatus for treating a human nail of a subject, the apparatus comprising:
a) an electromagnetic energy source operative to produce electromagnetic energy having a frequency of at least 0.5 MHZ (megahertz) and less than 10 GHZ (gigahertz), the electromagnetic energy source configured to produce an output electromagnetic signal (i.e. a signal of the ‘high frequency’ electromagnetic energy having a frequency of at least 0.5 MHZ (megahertz) and less than 10 GHZ (gigahertz)) directed to a conductive conformable applicator contactable with an upper surface of the nail of the subject;
b) a pulse controller operative to cause the electromagnetic energy source to deliver the output electromagnetic signal as a pulsed signal having one or more pre-determined pulse parameters, the pulse controller operative to effect a pulse-width modulation of the pulsed signal;
c) an impedance transformer operative to convert an impedance of tissue associated with the nail so that an intensity of reflected power (i.e. power delivered from the applicator/coupler and reflected from the upper surface of the nail plate) of the pulsed output signal is at most 0.1 an intensity (preferably, at most 0.02 or at most 0.01 an intensity) of incident electromagnetic power delivered from the applicator to an upper surface of the nail; and
d) the conductive conformable applicator for delivering the electromagnetic pulsed output signal to the nail, the conformable applicator configured to conform to a shape of the upper surface of the nail plate upon contact with the nail,
   wherein the apparatus is operative such that the delivery of the pulsed signal is effective to heat at least one of a nail plate tissue and nail bed tissue below the nail plate.

According to some embodiments, the delivery of the electromagnetic output signal is operative to induce a current flow through at least one of the nail plate, the nail bed, and nail matrix tissue of the nail, the apparatus further comprising: e) a ground electrode for receiving current of the current flow.

According to some embodiments, the apparatus lacks a ground electrode.

It is now disclosed for the first time apparatus for treating a human nail of a subject, the apparatus comprising:
a) an electromagnetic energy source operative to produce electromagnetic energy having a frequency of at least 0.5 MHZ (megahertz) and less than 10 GHZ (gigahertz), the electromagnetic energy source configured to produce an output electromagnetic signal (i.e. a signal of the ‘high frequency’ electromagnetic energy having a frequency of at least 0.5 MHZ (megahertz) and less than 10 GHZ (gigahertz)) directed to a conductive conformable applicator contactable with an upper surface of the nail of the subject;
b) the conductive conformable applicator for delivering the electromagnetic pulsed output signal to the nail, the conformable applicator configured to conform to a shape of the upper surface of the nail plate upon contact with the nail,
c) a delivered-energy localizer for localizing delivered energy of the electromagnetic pulse output signal to a pre-determined energy zone associated with a sharp thermal gradient such that upon delivery of the electromagnetic pulse output signal, at least one location with the pre-determined energy zone is heated to at least 50 degrees Celsius and the sharp gradient having a temperature variation in a location below the surface of the nail plate of at least 20 degrees Celsius/millimeter over a distance of at least 0.1

It is noted that apparatus for providing the delivered-energy localization may be implemented using any combination of electric components, electronic circuitry, software and mechanical components suitable for localizing the delivered energy to the pre-determined energy zone.

According to some embodiments, the delivered-energy localizer includes a pulse controller operative to cause the electromagnetic energy source to deliver the output electromagnetic signal as a pulsed signal having one or more pre-determined pulse parameters, the pulse controller operative to effect a pulse-width modulation of the pulsed signal.

According to some embodiments, the delivered-energy localizer includes an impedance transformer operative to convert an impedance of tissue associated with the nail so that an intensity of reflected power of the pulsed output signal is at most 0.1 an intensity of incident electromagnetic power delivered from the applicator to an upper surface of the nail.

According to some embodiments, the delivered-energy localizer includes a phase shifter operative to control a phase of the traveling waves of the pulsed output signals so that a location of maximum amplitude is substantially at the upper surface of the nail plate within a tolerance that is at most 0.5 a thickness of the nail plate.

According to some embodiments, the delivered-energy localizer includes e) a resonator located in the applicator, the resonator operative to cyclically accumulate and release a desired amount of energy to the nail via the applicator.

According to some embodiments, a location of a midpoint of the distance of the sharp gradient is substantially at a nail plate-nail bed interface within a tolerance that is at most 0.5 a thickness of the nail plate.

According to some embodiments, the tolerance is at most 0.2 the thickness of the nail plate.

According to some embodiments, the delivered-energy localizer is operative to localize the delivered energy such that the sharp temperature gradient is formed when the nail plate has a thickness of between 0.5 mm and 1 mm.

According to some embodiments, the delivered-energy localizer is operative to localize the delivered energy such that the sharp temperature gradient is formed when the nail plate has a thickness of between 1 mm and 3 mm.

According to some embodiments, the delivered-energy localizer is operative to localize the delivered energy such that the sharp temperature gradient is formed when the nail plate has a thickness of between 3 mm and 5 mm.

It is now disclosed for the first time a method of effecting a treatment of a nail condition, the method comprising:
a) identifying a patient suspected of having a nail having at least one malicious condition selected from the group consisting of a nail infection, a nail inflammation, and a nail deformation;
b) providing an electromagnetic energy delivery device having an energy delivery surface, the electromagnetic energy delivery device operative to deliver at least one high frequency electromagnetic energy selected from the group consisting of HF energy, RF energy and microwave energy;
c) positioning the energy delivery device to delivery sad high frequency electromagnetic energy to the nail;
d) delivering a sufficient high frequency electromagnetic energy dose (i.e. of electromagnetic energy having a frequency of at least 0.5 MHZ (megahertz) and less than 10 GHZ (gigahertz) (i.e. sufficient total energy and/or sufficient amplitude and/or sufficient time of treatment) from the energy delivery device to the nail to treat the malicious condition.

According to some embodiments, the treated malicious condition is the nail infection, and the nail infection is a fungal nail infection.

According to some embodiments, the treated malicious condition is the nail inflammation.

According to some embodiments, the treated malicious condition is the nail deformation.

According to some embodiments, the method further comprises:
d) before the delivery of the energy, subjecting the nail to a pre-treatment process which increases a nail plate water concentration of the nail;

According to some embodiments, the subjecting including subjecting the nail to a chemical agent which increases the nail plate water concentration of the nail.

### Fungal Nail Infection

According to some embodiments, the chemical agent includes urea.

According to some embodiments, the positioning includes coupling an applicator of the energy delivery device to an upper surface of a nail plate of the nail.

According to some embodiments, the applicator is a conformable applicator that changes conformation upon the coupling to the upper surface.

According to some embodiments, the method further comprises: d) determining a nail thickness; and e) determining the energy dose in accordance with the nail thickness.

According to some embodiments, the delivered energy includes pulsed-energy, and the method further comprises: d) forming an output signal of the pulse-energy in accordance with pre-determined pulse parameters.

According to some embodiments, the formed output signal includes a plurality of pulses, each having a pulse duration of between 10 nanoseconds and 10 milli seconds (for example, between 1 and 100 microseconds).

According to some embodiments, the method further comprises: d) employing a phase shifter to alter an electromagnetic output signal of the energy device to concentrate delivered energy in pre-determined energy zone.

According to some embodiments, the method further comprises: d) converting impedance of biological tissue (i.e. nail plate and/or nail matrix and/or nail bed) associated with the treated nail to a corrected value so that an output signal of the energy delivery device passes through an upper surface of a nail plate of the nail without substantially being converted to a standing wave.

According to some embodiments, the method further comprises: the energy delivery device includes a resonator, the method further comprising: d) cyclically accumulating and releasing a desired amount of energy in the resonator.

According to some embodiments, the method further comprises: the delivering is effective to heat at least one of a nail plate tissue and nail bed tissue below the nail plate to a temperature sufficient to inhibit activity of a pathogen residing within the heated tissue.

According to some embodiments, the method further comprises: the delivering is effective to heat at least one of a nail plate tissue and nail bed tissue below the nail plate to a temperature sufficient to inhibit activity of a fungal pathogen residing within the heated tissue.

According to some embodiments, the method further comprises: the delivering is effective to heat at least one of a nail plate tissue and nail bed tissue below the nail plate to at least 50 degrees Celsius

According to some embodiments, the method further comprises: the delivering of the energy is effective to produce a sharp temperature gradient having a temperature variation of at least 20 degrees/millimeter over a distance of at least 0.1 mm in at a location below the surface of the nail plate

It is now disclosed for the first time a method of treating or ameliorating an infectious in a subject, the method comprising: a) coupling an energy delivery surface (for example, a surface of an applicator/coupler) of an electromagnetic energy delivery device with a hard tissue; and b) delivering electromagnetic energy from the energy delivery device to the hard tissue to heat biological tissue of the subjecting including at least a portion the hard tissue in a manner that is effective to treat or ameliorating the infectious condition (i.e. killing a pathogen of the infectious condition, or reducing the pathogen population, or reducing the activity of existing pathogens) in the subject.

According to some embodiments, the hard tissue is keratin-rich hard tissue (i.e. hard tissue that comprises at least 10%, or at least 25%, or at least 50% keratin) (for example, fingernails or toenails).

According to some embodiments, the hard tissue is non-mineralized hard tissue (for example, hard tissue other than bones, for example, fingernails or toenails).

According to some embodiments, the energy delivery surface is a conformable surface which conforms to a shape of a surface of the hard tissue upon the coupling.

According to some embodiments, the delivered electromagnetic energy is selected from group consisting of HF energy, RF energy and microwave energy.

According to some embodiments, the delivered electromagnetic energy is effective to produce a sharp temperature gradient having a temperature variation of at least 20 degrees/millimeter over a distance of at least 0.1 mm in at a location below the surface of the nail plate

According to some embodiments, the delivered energy is effective to heat at least a portion of the biological tissue to at least 60 degrees Celsius.

According to some embodiments, the method further comprises: c) before the stage of delivering, pre-treating the hard tissue with an agent that modifies electromagnetic energy absorbance properties.

According to some embodiments, the method further comprises: c) before the stage of delivering, pre-treating the hard tissue with an agent that modifies mechanical properties of the hard tissue.

According to some embodiments, the method further comprises: c) before the stage of delivering, pre-treating the hard tissue with an agent that softens at least one of the hard tissue and biological tissue below the hard tissue.

According to some embodiments, the method further comprises: c) before the stage of delivering, pre-treating the hard tissue with an agent that increases a water content of the hard tissue.

According to some embodiments, the method further comprises: c) before the stage of delivering, pre-treating the hard tissue with an agent comprising urea.

According to some embodiments, the electromagnetic energy is delivered without removing or cutting the hard tissue.

According to some embodiments, the method further comprises: c) before the energy delivery, measuring at least one geometric parameter of the hard tissue (for example, a thickness); and d) computing at least one energy delivery parameter in accordance with results of the measuring.

According to some embodiments, the delivered energy is effective to treat the infectious condition by killing at least a portion of a population of pathogens associated with the infectious condition.

According to some embodiments, the method further comprises: the killing includes killing spores of the population of pathogens.

According to some embodiments, the method further comprises: the killing includes killing dormant pathogens.

According to some embodiments, the method further comprises: the population of pathogens include fungus.

These and further embodiments will be apparent from the detailed description and examples that follow.

### BRIEF DESCRIPTION OF THE DRAWINGS

FIGS. 1A-1C depict a finger or toe nail and exemplary temperature profiles in accordance with some embodiments of the present invention.

FIGS. 2, 5-6, 7A-7B depicts a schematic of an exemplary device for delivering electromagnetic radiation to nail tissue in accordance with some embodiments of the present invention.

FIG. 3 depicts an operation of an exemplary conformable applicator (coupler) in accordance with some embodiments of the present invention.

FIGS. 4A-4B depict an exemplary device for delivering electromagnetic radiation to nail tissue in accordance with some embodiments of the present invention.

FIGS. 8A-8B depict different shaped applicators (couplers) in accordance with different embodiments of the present invention.

FIG. 9 depicts a schematic of an exemplary device for delivering electromagnetic radiation to nail tissue in unipolar and/or bipolar modes in accordance with some embodiments of the present invention.

FIG. 10 illustrates a flow-chart of an exemplary method for treating nail conditions and/or for operating a device in accordance with exemplary embodiments of the present invention

FIG. 11 provides graphs illustrating some experimental results.

While the invention is described herein by way of example for several embodiments and illustrative drawings, those skilled in the art will recognize that the invention is not limited to the embodiments or drawings described. It should be understood that the drawings and detailed description thereto are not intended to limit the invention to the particular form disclosed, but on the contrary, the invention is to cover all modifications, equivalents and alternatives falling within the spirit and scope of the present invention. As used throughout this application, the word “may” is used in a permissive sense (i.e., meaning “having the potential to’), rather than the mandatory sense (i.e. meaning “must”).

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice.

The present inventors are now disclosing apparatus and methods for treating, with high frequency electromagnetic radiation (e.g. HF, RF/shortwave and/or microwaves), nails (for example, infected nails), hard tissue, and tissue which usually has a low water concentration. The presently disclosed apparatus and methods are particular useful for treating nail fungus.

Through this disclosure, the term “high frequency radiation” or “high frequency electromagnetic radiation” refers to short wave radiation (RF) (for example, RF and/or HF and/or microwave radiation having a frequency of at least 0.5 MHZ (megahertz) and less than 10 GHZ). High frequency radiation also includes so-called HF radiation having a frequency between 0.5 kHz and 220 MHZ. In one example, high frequency radiation refers to short wave radiation in the 2-100 MHz range, for example, 13.56, 27.12, 40.68 MHz ISM frequencies. In another example, high frequency radiation refers to microwave radiation, for example, for example, having a frequency between 0.87 and 2.45 GHz (for example, 0.87 or 2.45 GHz).

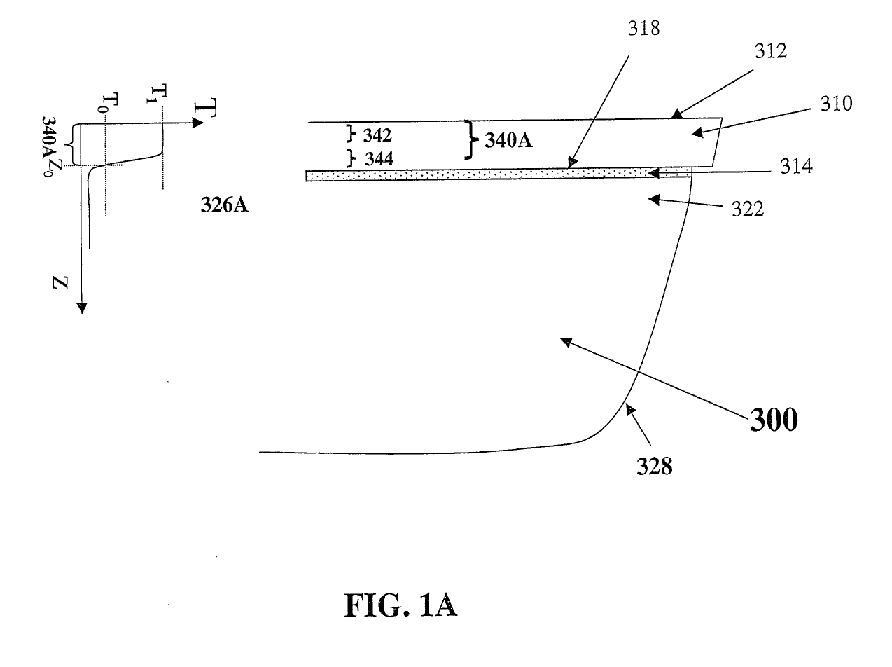

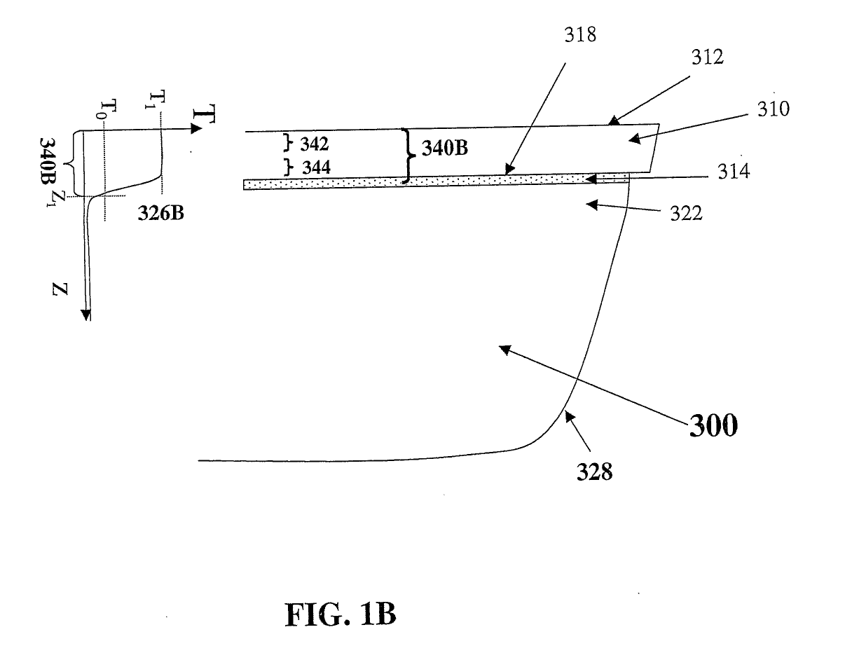

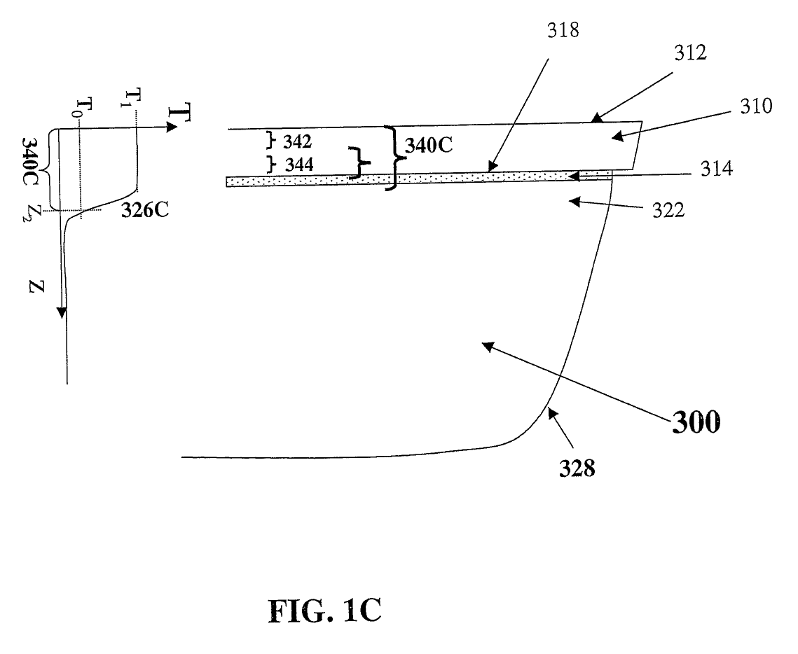

Heating of Nail Plate Tissue and/or Nail Bed Tissue (Discussion of FIGS. 1A-1C)

FIGS. 1A-1C (not drawn to scale) provide an illustration of a finger or toe 300, including a nail plate 310 having an upper surface 312, and a nail bed below the nail plate having upper 314 and lower regions 322. The nail bed includes epidermis tissue 314 and a sub-dermal tissue 322 (i.e. the dermis, sub-dermis, etc). At the interface 318 between the nail plate 310 and the upper surface of the epidermis 314 of the nail bed there is typically connective tissue (not shown) between the upper surface of the nail bed (i.e. the upper surface of the epidermis) and the lower surface of the nail plate 310.

In exemplary embodiments, high frequency energy is applied to the nail plate 310 and/or the nail bed (e.g. via the upper surface 312 of the nail plate 310). This wave is effective to generate a sharp temperature gradient at a selected location beneath the upper surface 312 of the nail plate (for example, the upper or lower half of the nail plate) and/or an upper portion of the nail bed. This sharp temperature gradient is useful for heating selected tissue to a temperature that is sufficient to reduce activity of the pathogenic fungus without concomitantly damaging (or with minimum damage) deeper layers of tissue below the nail plate. As will be explained below, different mechanisms for achieving this temperature gradient are disclosed, for example, by selecting certain pulse parameters and/or by providing a phase shifter for controlling a location of a maximum amplitude of a traveling electromagnetic wave.

Referring once again to FIGS. 1A-1C, it is noted that fungal infection of the nail may be associated with a presence of pathogenic fungus within the nail plate 310 and/or the nail bed epidermis 314 and/or certain regions of the nail bed dermis 322. In exemplary embodiments, the sharp temperature gradient is created (see 326A of FIG. 1A, 326B of FIG. 1B, and 326C of FIG. 1C) such that certain regions heated to a “high temperature”) i.e. a temperature high enough to inhibit an activity of pathogens) while other (typically deeper regions) are heated to a lesser extent, or not at all, and thus, are associated with a “low temperature.” It is appreciated that in many clinical situations, there may be a trade-off between aggressiveness of treatment of the fungal infection and the desire to not damage deeper tissue layers and/or inflict pain on the patient. In FIG. 1A, an exemplary “less aggressive temperature profile” is illustrated, while in FIG. 1B, an exemplary “more aggressive temperature profile” is illustrated, while in FIG. 1C, an exemplary “even more aggressive temperature profile” is illustrated. In exemplary embodiments, at least a portion of the nail plate 310 and/or optionally at least a portion of the nail bed epidermis 314 and/or optionally a portion of the sub-dermal tissue 322 (dermis, etc) is heated to a “treatment temperature”—for example, between 50 degrees and 80 degrees, or between 60 degrees and 70 degrees. For the specific case of fungal infection of the nail, heating to the “treatment temperature” may be useful for reducing or eliminating fungus pathogen activity and/or for reducing a population of fungus pathogens (for example, by killing the pathogen, for example, killing fungus spores and/or plants).

The specific temperature and duration of treatment required may also depend on the exact pathogen species and other parameters related to patient physiology.

For embodiments related to fungal infections of the nail, the main target of heating is typically the upper portion of the nail bed (for example, the epidermis 314 and optionally an upper region of dermis tissue 322) and the underlying part of the nail plate 344, where fungus pathogens reside.

The skilled artisan will, in accordance with relevant clinical parameters, be able to select parameters that produce the desired temperature profile. For the non-limiting exemplary device described below, the temperature profile may be controlled in accordance with one or more pulse parameters and/or phase shift parameters, for example, a pulse amplitude and/or duration and/or frequency. For example, as explained below, short intense pulses may be used for producing a sharp temperature gradient. Not wishing to be bound by theory, it is noted that short acting pulse(s) may be useful for localizing delivered energy because the rate of energy is much faster than a rate of energy diffusion, thereby creating a strong gradient.

In exemplary embodiments, phase shifter is also used to control a location of the maximum of the traveling electromagnetic wave delivered to the nail, thereby controlling the temperature profile. In exemplary embodiments, the location of maximum intensity is selected at or near (i.e. within a millimeter or, or within a few millimeters) the upper surface 312 of the nail plate 310.

Other factors influencing the temperature profile may include the relative thermal conductivity of the nail plate tissue and nail bed tissue, and the water concentration of the nail plate tissue. Another factor which may, in various embodiments, influence the temperature profile is the circulation of bodily fluids in, for example, blood and lymphatic vessels, which may provide convective cooling in one or more layers of sub-dermal tissue of the nail bed, thereby confining the sharp temperature to the nail plate and/or the nail bed epidermis

It is noted that because human nails typically have a relative low water content, in some embodiments, the nail is pre-treated (for example, with a chemical agent such as a urea-containing formulation; for example, a single pre-treatment or several pre-treatments, for example, at least 24 hours before delivery of the high-frequency electromagnetic radiation) to increase the water concentration of the nail plate. Not wishing to be bound by theory, it is noted that the pre-treatment process is important in some embodiments because it may dramatically increase the water concentration in the nail plate 310, for example, up to 40-60%, and therefore facilitate a process of energy absorption in the keratin-rich nail plate tissue which sustains fungus pathogens.

As noted earlier, the exact temperature profile may vary from patient to patient, in accordance with device parameters and/or biophysical parameters of the treated region. In exemplary embodiments, the delivered high-frequency radiation produces a sharp temperature gradient having a temperature variation of a minimum gradient value that is 10 degrees/millimeter over a “minimum gradient distance” of at least 0.2 mm in a “sharp gradient region” below said nail surface. In some embodiments, the “minimum gradient value” is at least 10, 20, or 30, or 40 degrees/millimeter, where the exact value may be controlled, for example, by device parameters selected by an attending care-giver, for example, in accordance with the patient's medical profile. This sharp temperature gradient may be generated in the nail plate 310 and/or the nail bed epidermis 314 and/or the nail bed dermis 322.

In some embodiments, the aforementioned temperature gradient having the minimum gradient value is generated for at least the minimum gradient distance in one or more of the following regions: the nail plate, or a region in proximity of the nail plate-nail bed interface, for example, less than a given value from the nail bed-nail plate interface (above the nail bed-nail plate interface in the nail plate, or below the nail bed-nail plate interface in the nail bed). In exemplary embodiments, this “given value” is at most 1.5 mm, or 1 mm, or 0.5 mm, or 0.2 mm. In exemplary embodiments, a ratio between this “given value” and a thickness of the nail plate is at most 1.5, 1, 0.5 or 0.2.

Optionally, a cooling process (pre-cooling and/or concomitant cooling) is effected, for example, by cooling a lower surface 328 of the finger and/or toe, or any other cooling process.

### Exemplary Device

Although various teachings of the present invention (for example, treating, with high-frequency radiation, hard tissue and/or a nail and/or an infection, such as a fungal infection; for example, using a conformable applicator) are relevant both for so-called multi-electro devices (i.e. including an electrode for delivering current and a ground electrode) and single-electrode (or “unipolar”) devices, various teachings will now be explained with reference to an exemplary non-limiting “unipolar” device with a single electrode or applicator that delivers a traveling wave of electromagnetic energy (e.g. HF, RF and/or microwave energy) via an upper surface of the patient's nail. The exemplary system/device thus lacks a ground electrode.

Thus, in exemplary embodiments, the exemplary system includes a single conformable HF, RF and/or microwave-energy applicator which serves as electromagnetic energy (e.g. HR and/or RF and/or microwave energy) coupler that functions in combination with an adjacent tissue (i.e. the nail plate and/or nail bed) as a losses transmission line when contacted with upper surface of the nail plate.

In some embodiments, exemplary system further includes a treated area (part) of the nail, in direct contact (via an upper surface of the nail plate) with a conductive conformable applicator where the underlying nail plate and optionally nail bed acts as a dissipative load of the said applicator and the transmission line.

In some embodiments, the exemplary system further includes a parallel resonance circuit (resonator—for example, RF, HF or microwave resonator) including inductor and capacitor and maximally closely contacted with the applicator by its central point where a merit factor of this resonant circuit may be sufficiently high to provide active losses determined by an equivalent resistance, which is, for example, at least 20 times higher that modulus of impedance of a targeted tissue (i.e. the nail plate and/or nail bed).

In some embodiments, the exemplary system further includes impedance matching network (IMN) converting the impedance of the treated nail plate and/or nail bed into 50 Ohms in order to minimize reflected power from human tissue and to provide a traveling wave through nail plate and/or nail bed tissue. In some embodiments, at most 3%, or at most 1%, or at most 0.5% of the delivered power reflected.

In exemplary embodiments, the system includes a phase shifting device at the input of the IMN to provide an achievement of maximum amplitude of an electromagnetic traveling-wave at a predetermined location relative to the upper surface to the nail—for example, at or near the upper surface of the nail, for example within a tolerance of a few millimeters.

In exemplary embodiments, the impedance transformer (for example, impedance matching network) facilitates organization of energy dissipation in an “energy dissipation zone”—for example, the nail plate and/or an “upper layer” of tissue in the nail bed—for example, the nail bed epidermis and/or a portion of the nail bed dermis. This may be useful for treating these energy dissipation zones (for example, treating a fungal infection) while concomitantly maintaining a relatively low temperature below the energy dissipation zone.

In exemplary embodiments, application of electromagnetic (e.g. RF and/or HF and/or microwave) power in short-pulses provides fast and effective heating of the targeted energy nail plate and/or portion of the nail bed with a relatively low average electromagnetic power level.

In some embodiments, the device includes a supporting parallel resonator, for example, attached in proximity to the applicator and operative to accumulate HF, RF and/or microwave energy to provide efficient excitation of the applicator and high electromagnetic voltage on the application surface.

As noted earlier, in some embodiments, the high frequency energy is delivered in a series of one or more pulses. Thus, in some embodiments, the device includes a mechanism for PWM-control of output RF-power (or HF or microwave power) and simple IMN techniques provide good matching and low RF/HF/microwave-power reflection with all types of human tissues. Not wishing to be bound by theory, it is noted that one possible advantage of applied fixed IMN and PWM-control is to ensure good impedance matching with variable types of nails without effecting a complicated impedance matching correction.

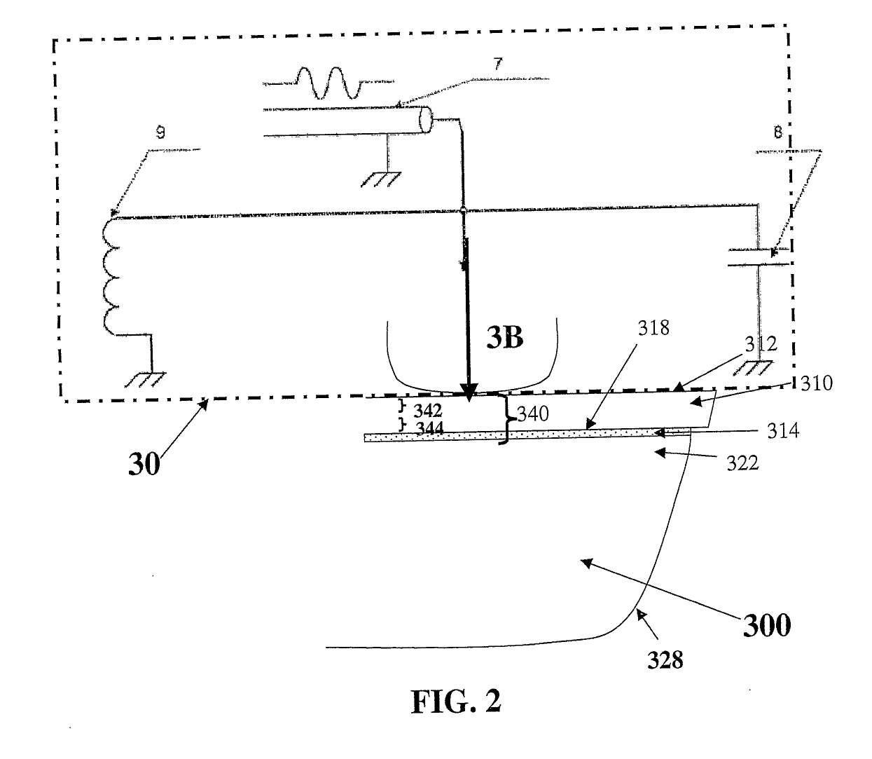

### Discussion of FIG. 2

FIG. 2A provides a schematic diagram of an exemplary energy delivery system for treating a nail plate and/or nail bed.

As illustrated in FIG. 2, system 30 includes a high frequency electromagnetic energy source 10 (for example, an RF/shortwave energy source and/or HF and/or a microwave energy source) capable of producing an output high-frequency power signal 17 to a conformable applicator 3 contactable with an upper surface 312 of a fingernail plate and/or toenail plate 310 belonging to the subject. Applicator 3 is capable of delivering a desired amount of energy to a predetermined energy dissipation zone 340 (see FIGS. 1A-1C), for example, including the nail plate 310 or a portion thereof and/or the nail plate or a portion thereof.

As shown in FIG. 2, system 30 further includes a phase shifter (e.g. trombone-type phase shifter 14). Phase shifter 14 is capable of shifting a phase of directed traveling waves of output power 17 so that energy therefrom is concentrated primarily in the predetermined energy dissipation zone 340. In one example, the energy dissipation zone 5 includes the upper surface 312 of the nail plate 310 and the maximum of the traveling wave is at or near the upper surface 312 of the nail plate 310.

In exemplary embodiments, system 30 further includes an impedance transformer (for example, an impedance matching network 11) (IMN capable of converting the nail plate and/or nail bed belonging to the subject from a nominal value (e.g. 250-500 Ohms) to a corrected value (e.g. 50 Ohms). The corrected value matches an impedance characteristic of RF (or HR or microwave) energy source and RF (or hr or microwave) transmission line including phase shifter 14 and cable 7, so that output RF traveling wave 17 may pass through surface 312 of the nail plate 310 without being converted to a standing wave.

In exemplary embodiments, system 30 further includes an RF resonator 13 located in conformable applicator 3. RF and/or microwave and/or HF resonator 13 is capable of cyclically accumulating and releasing the desired amount of energy and is further capable of concentrating the desired amount of energy so that a significant portion thereof may be transmitted to predetermined energy dissipation zone 340.

System 30 further includes conductive, conformable applicator 3 capable of conveying the output electromagnetic power signal 17 from the RF and/or microwave and/or HF energy source 10 through surface 312 of the nail plate 310 to the predetermined energy dissipation zone 340 after output 17 has been processed by the phase shifter 14, IMN 11 and resonator 13.

Typically, operation of system 30 results in 2 to 4% loss of energy from output signal 17 with ban additional 2-4% reflection of energy from output signal 17. This means that system 30 can reliably deliver 90-95% of energy from output signal 17 into zone 340. Neither concentration of energy from output signal 17 into a small zone 340, nor this degree of efficiency, were achievable with previously available alternatives. IMN 11 reduces reflection of applied output signal 17 from surface 6 thereby increasing efficiency of delivery of energy to energy dissipation zone 340.

It is noted that for the particular example described in the figures, the absence of a ground electrode from system 30 permits free propagation of the waves of output RF and/or HF and/or microwave power signal 17 in energy dissipation zone 340.

These factors serve to greatly increase the degree of heating of water molecules 1 relative to the degree of heating of water molecules 1 achieved with prior art alternatives.

In some embodiments, system 30 further includes a pulse controller (for example, a “PWM controller”), where the PWM controller 12 is capable of causing the electromagnetic energy source to deliver the output power signal in pulses of a predetermined duration and amplitude with a desired frequency. In one particular example, the operating electromagnetic “radiation frequency” is 40.68 MHz, a PWM-frequency or “pulse frequency” is 20 Hz to 100 kHz, and a duty cycle is 1 to 100%, for example, from 1-10%, or from 1-25% or from 10-90%. In exemplary embodiments, pulse duration is between 2 microseconds and 100 microseconds though shorter and/or longer pulses are both within the scope of the invention.

The operating power is in the targeted area. In one particular example, the peak power per pulse is between 200 and 600 watts. The average power depends on the duty cycle, and may be, for example, between 10 and 50 watts.

In some embodiments, the peak and/or average power is selected in accordance with a measured nail thickness. In one example, if a nail is thicker, a higher amplitude pulse and/or longer pulse duration and/or higher PWM frequency is selected.

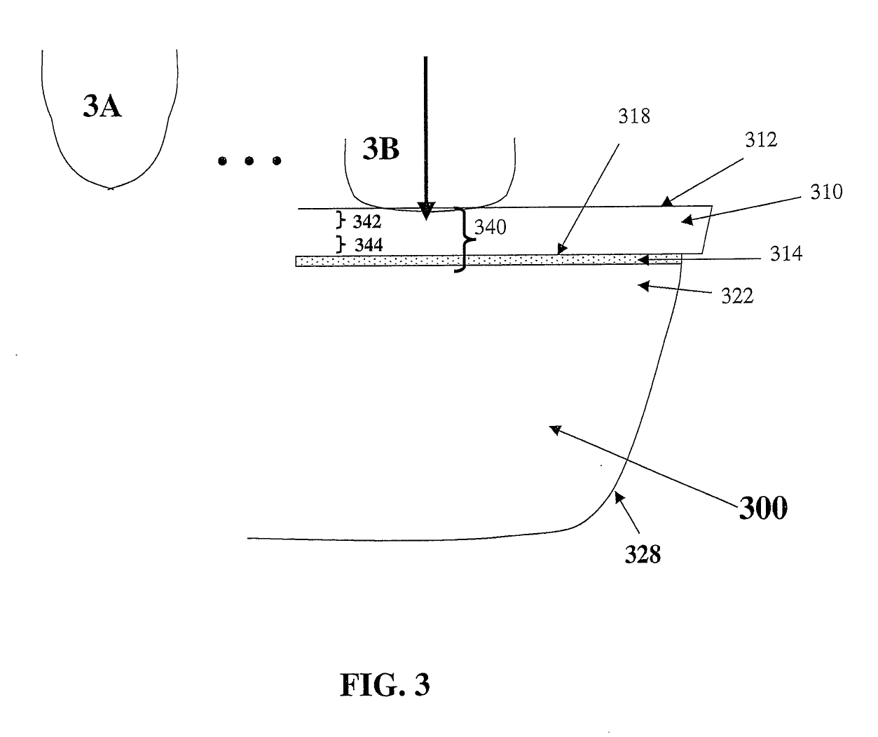

The Conformable Applicator (Discussion of FIGS. 3; 4A-4B)

FIG. 3 illustrates a process whereby a soft, conformable applicator 3 is contacted with a relatively hard upper surface 312 of the nail plate 310.

When pressed (for example, gently pressed) down onto the nail, the applicator 3 “conforms” from configuration 3A to 3B to adopt a shape whereby a lower surface of the applicator “fits” the upper surface 312 of the nail plate 310. Not wishing to be bound by theory, it is noted that the conformable applicator 3 is useful for treating different nails, each nail having a different shape and size.

It is noted that conducting applicator 3 serves as an electrode for delivery of the high-frequency electromagnetic energy to the nail plate 310. As such, in order for the applicator surface to provide a “tight contact” with nails of different size and shape, allowing the energy applicator 3 to server as a coupling element that functions in combination with the nail plate as a substantially lossless transmission line where the biological matter (i.e. nail plate and/or nail bed) dissipates a traveling wave delivered via that applicator 3.

Thus, in order to provide this “good contact,” it may be advantageous to use an applicator with a surface that “conforms” to the human nail surface, for example, a soft applicator that conforms to the shape of the hard upper surface of the human nail.

There is no explicit limitation on the material from which applicator 3 may be constructed, though in exemplary embodiments, the conformable applicator is soft and/or elastic and conducting. Appropriate materials from which the applicator/coupler may be constructed include but are not limited to rubber, elastometric material, knitted materials, string, strips, etc. In one example, the applicator/coupler includes flexible metallic fibers. If the applicator is constructed primarily from a material not sufficiently conductive for the applicator to function as an electrode (for example, rubber, which is constructed from insulating rubber), it is possible to associate a second more conducting material with the insulating conformable (for example) so material. For example, conducting pieces of material (for example, metal) may be embedded in a conformable, soft, less conducting matrix. Nevertheless, other embodiments are contemplated, for example, a soft conducting polymer.

In some embodiments, the conformable applicator/coupler includes a dielectric coating for preventing of occasional touching between the applicator/coupler and soft tissues. The coating may be implemented using, for example, rubber-sprayed cover, latex, etc.

The applicator may be any shape, including but not limited to pyramidal conical, spherical, hemispherical, and cylindrical.

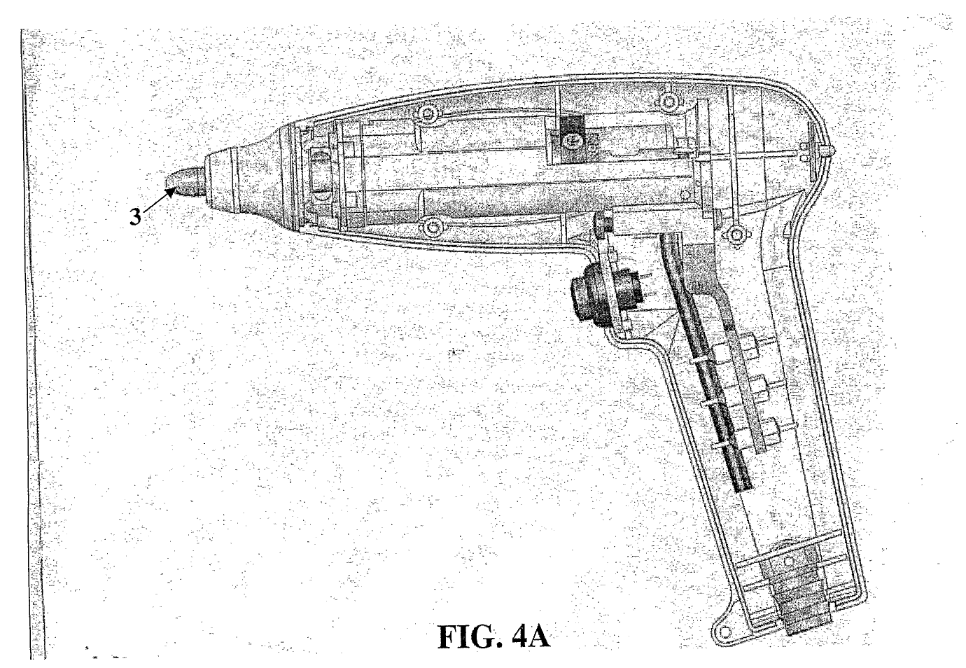

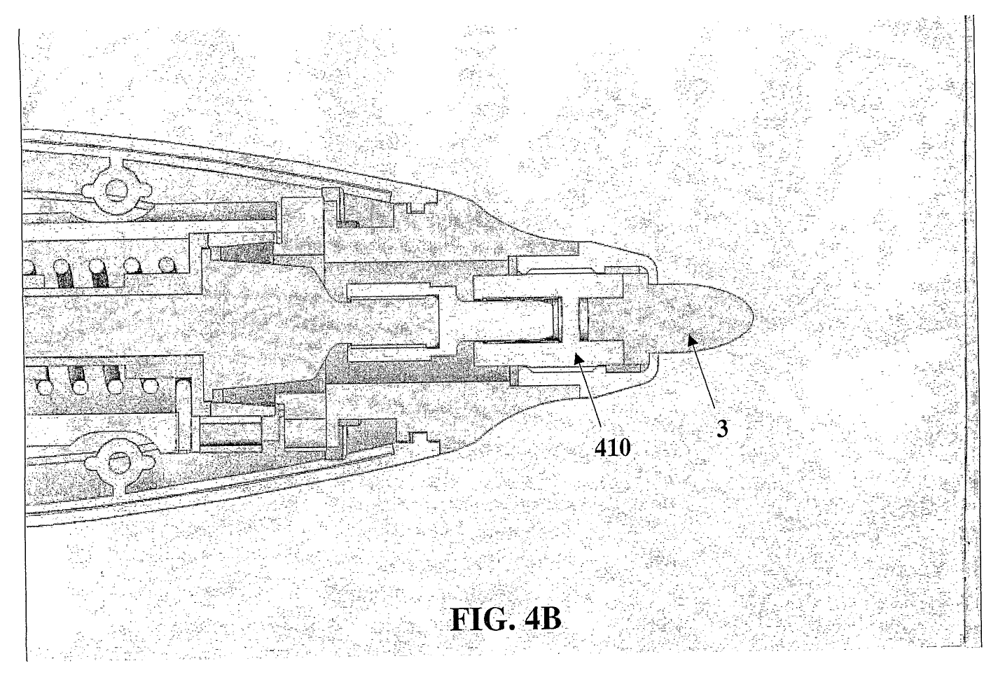

FIGS. 4A-B illustrate an exemplary energy delivery device including a soft applicator 3 associated with the housing via a holder 410 for example, a metal holder 410.

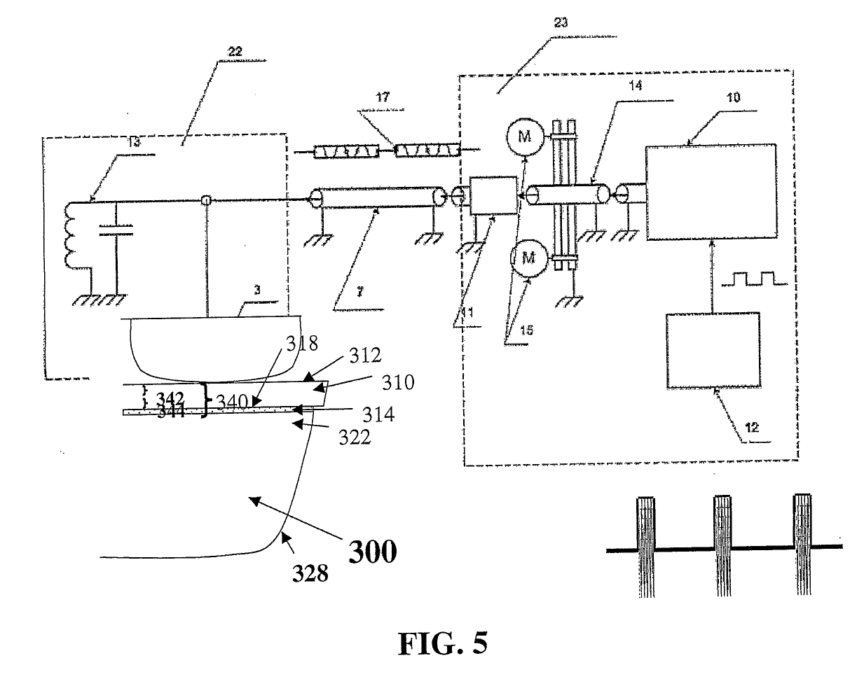

### Discussion of FIG. 5

Optionally, but preferably, system 30 further includes a feeding cable 7, the feeding cable 7 connecting electromagnetic energy applicator 3 and the electromagnetic resonator 13 with IMN 11. Optionally, but preferably, feeding cable 7 has a resonance length defined by n*λ/2 length, where λ is a wavelength of electromagnetic energy in the cable material and n is a whole number.

Optionally, but preferably, IMN 11 includes a fixed structure characterized by a shape such as, for example, L shaped, T shaped or Π-shaped structure. Optionally, but preferably, IMN 11 includes a broadband impedance transformer. Optionally, but preferably, IMN 11 is variable.

Optionally, but preferably, phase shifter 14 includes a trombone type phase shifting mechanism.

Alternately, or additionally, phase shifter 14 may be at least partially constructed of coaxial cable.

Optionally, but preferably, phase shifter 14 provides a variable phase shift.

Optionally, but preferably, RF-generator 10 delivers coupled energy.

Optionally, but preferably, the energy delivered to the predetermined energy dissipation zone 5 is coupled in a continuous or a pulsing mode. RF-generator 10 produces output 17 in the form of a sinusoidal signal which may be modulated by rectangular pulses with lower frequency. This may be accomplished, for example, by PWM controller 12.

According to some embodiments, system 30 employs RF-energy 17 which is characterized by a resonance frequency that matches a known natural resonance frequency of the selected target.

Optionally, but preferably, system 30 may further include additional components, such as, for example, a laser beam, an ultrasonic transducer, a UV light source, a plasma treatment device and a flash lamp.

As shown in FIG. 5, in order to maximize transmission of RF-energy from RF-source 10, through resonant cable 7 to applicator 3, it is connected to the central point of a parallel resonator 13 including a capacitor 8 and an inductor 9 connected in parallel and characterized by very high-Q-factor for example more than 20.

In exemplary embodiments, the delivered oscillating RF-power (e.g. 25-300 watts) is stored in the resonator 13 therefore an active (dissipative) load of resonator 13 is only an adjacent tissue.

In exemplary embodiments, the active losses of resonator 13 are very low (20-50 times less than energy dissipated inside tissue 4). The intermittent discharge of capacitor 18 and the inductor 19 is coupled through applicator 3 to tissue 4.

In one particular example, RF-generator 10 capable of producing 200-400 Watts full power at 40.68 MHz operating frequency demonstrates an optimal performance at 50 Ohms resistive load. Optimal performance means minimal reflected RF-power with maximum RF-forward power dissipated by a load. Thus, the real load that includes treated volume 340 of nail plate and/or nail bed tissue may be matched as a 50 Ohms load.

The used impedance matching network (IMN 11; see FIG. 5) is fixed (the elements are not variable operatively); therefore operation occurs without RF and/or HF and/or microwave power amplitude changes. The control of output power is reached by PWM-control (pulse width modulation). PMW achieves modulation of output power by rectangular power pulses applied with a frequency lower than that of the RF wave. Decreasing RF and/or microwave-power coupling may be carried out by reducing a duty cycle of PWM. PWM-controller 12 produces rectangular pulses with a modulation frequency range of 20 Hz-100 kHz. In exemplary embodiments, duty cycle may be varied from 5 to 100%. The shape of an exemplary RF and/or HF and/or microwave power pulse 17 is showed in FIG. 5. The PWM-control of output RF and/or HF and/or microwave power permits high peak RF and/or HF and/or microwave power level in heating zone 340 with lower average power

In some embodiments, the energy for heating the dissipation zone 340 will reach over 80% (or higher—for example, over 90%) of total energy delivered to the biological, and the remaining energy is dissipated through diffusion of the heat to the surface and/or by convection of natural liquids of human body.

As illustrated in the figures, applicator 3 is connected to parallel resonator 13. Applicator 3 and resonator 13 are physically positioned inside of operating hand-piece 22 used for treatment procedure. The IMN-system locates inside the main system 23. In order to avoid a mismatching phase shift between the applicator 3 and the IMN 11, the length of cable 7 is equal to the whole number (n) of wavelength of RF and/or HF and/or microwave-energy (λ) in the cable material.

In some embodiments, a phase shifting system (e.g. trombone-type system 14) is inserted between output of RF and/or HF and/or microwave-generator 10 and an input of IMN 11. The position of the maximum of energy dissipation can be controlled by this phase shifting system and can be placed, for example, on or near the upper surface 312 of the nail plate. In order to control the depth of RF and/or HF and/or microwave-energy penetration the length of trombone can be shortened that change a position of dissipated electromagnetic wave in the tissue or an area of the maximum of electromagnetic voltage, typically, at or near the upper surface 312 of the nail plate

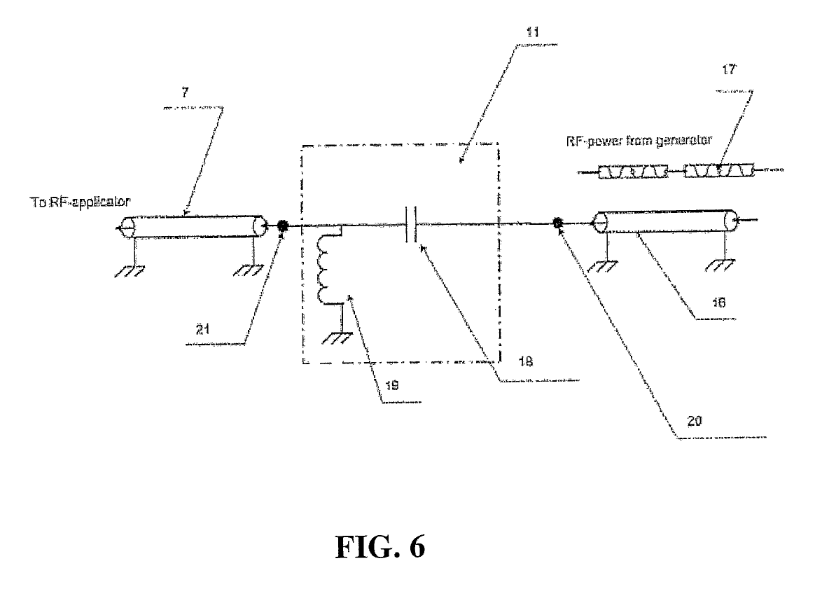

In exemplary embodiments, phase shifter 14 can be controlled automatically, for example by motors 15. This change of phase could be linear or periodical that influences a heat penetration depth. One exemplary practical implementation of impedance matching system 11 is illustrated by FIG. 6. According to this example, the RF, HF and/or microwave power delivered from RF, HF and/or microwave generator 10 through coaxial cable 16 is modulated by rectangular pulses 17. Because RF-generator 10 is matched to 50 Ohms and impedance of human tissue is close to 300-450 Ohms, according to this example, it is necessary to convert 50 to 300-450 Ohms with compensation of electromagnetic reactance of tissue 4. According to this example, this is achieved with impedance matching network 11. L-type simple fixed IMN consisting of RF-capacitor 18 and RF-inductor 19 (FIG. 6) were applied for this purpose. According to this example, half-wave cable 7 is applied for transmission of RF and/or HF and/or microwave-energy from IMN 11 to RF and/or HF and/or microwave applicator 3 without phase shifting that is controllable by phase shifting system. According to this example, the measured impedance in the point 20 is 50 Ohms and in the point 21 is 300-450 Ohms. Impedance matching networks of various types may be employed without significantly altering performance of the present invention. Regardless of the exact IMN type employed, the IMN 11 can be variably and/or automatically controlled to trace an impedance changing.

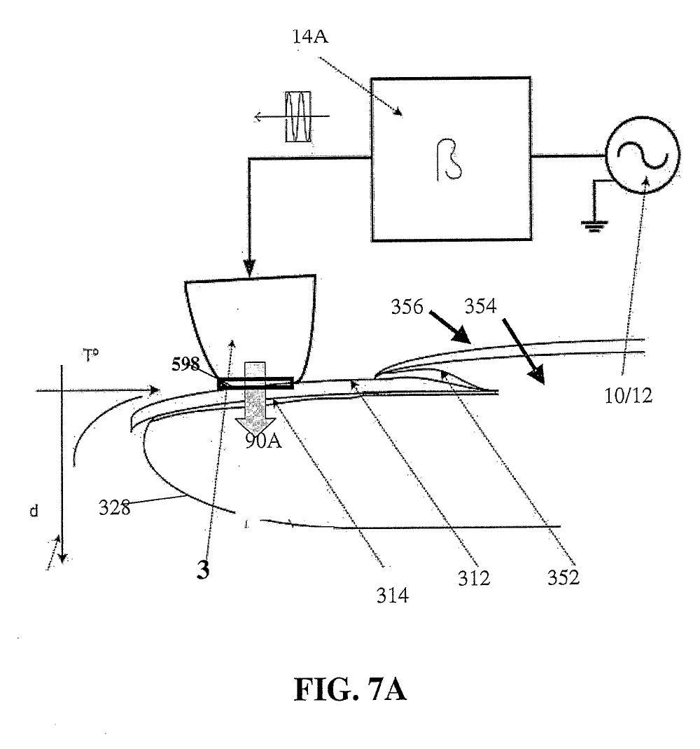

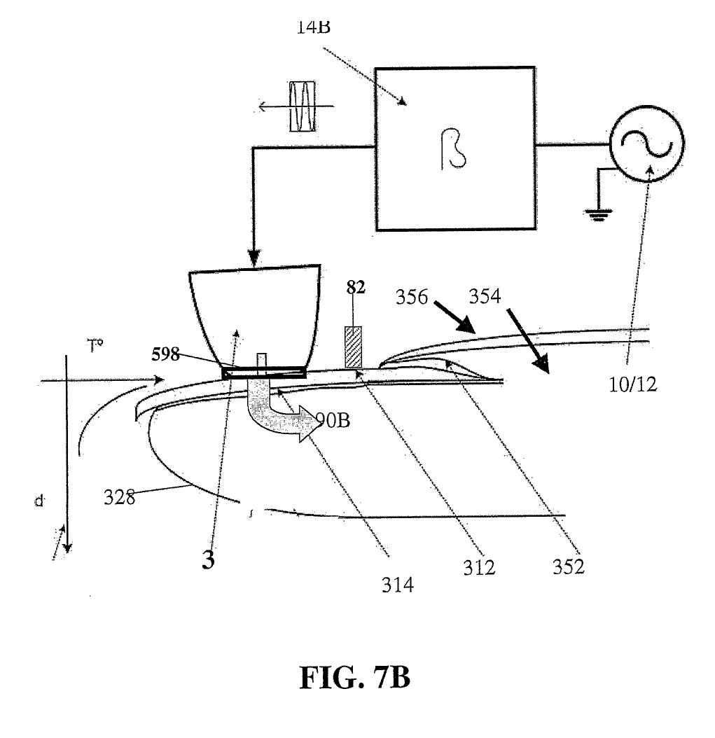

It is noted that the teachings of the present invention are applicable both the so-called unipolar devices which lack a return ground electrode (i.e. devices where the patient's body functions as an antenna) as well as bipolar devices which provide a ground plane electrode 82. FIGS. 7A-7B illustrate exemplary unipolar (FIG. 7A) and bipolar (FIG. 7B) configurations in accordance with exemplary embodiments of the present invention. FIGS. 7A-7B also depict the nail matrix 356 as well as other adjacent tissue 354.

Referring to FIG. 7A, it is noted that electrical current flows substantially perpendicularly 90A to a local contact region of the upper surface 312 of the nail plate. In contrast, in the exemplary bipolar system of FIG. 7B, the presence of the ground electrode 82 causes the electrical current to flow as illustrated in 90B. The ground electrode 82 may be located in various locations, including locations where the nail matrix 356 and/or other tissue adjacent to the nail 354 and/or tissue beneath gets heated.

It is noted that as illustrated in FIGS. 7A-7B, the coupling between the applicator/coupler 3 and the upper surface 312 of the nail plate 310 is a “tight coupling” 598. It is noted that the conformable (for example, soft) applicator which conforms to a shape of the upper surface of the nail to provide the right contact. This tight contact reduces energy losses during energy delivery, providing for a more efficient treatment.

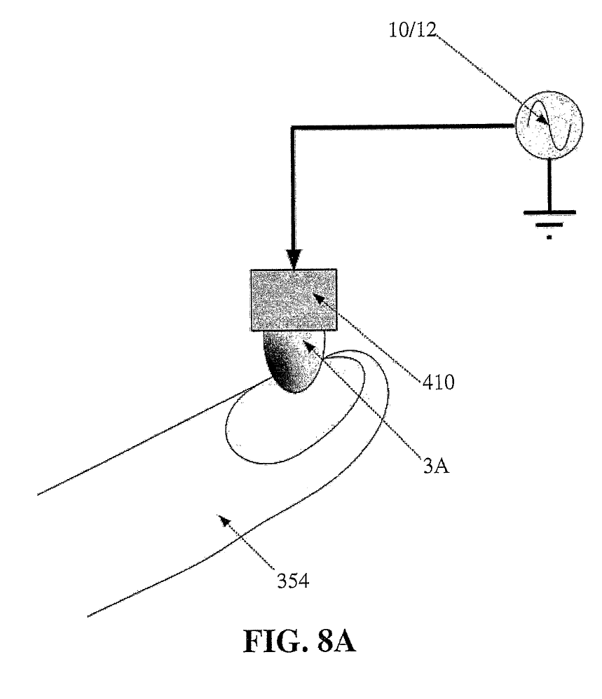

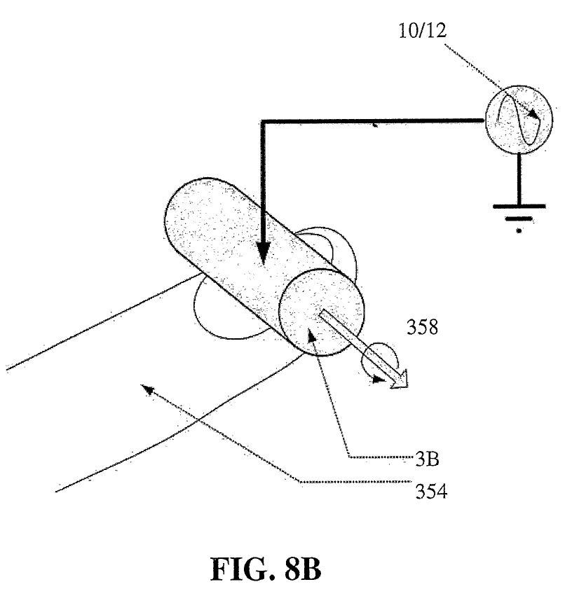

It is noted that there is no limitation on the shape of the applicator 3. Thus, FIGS. 8A-8B illustrate two exemplary applicators 3, both the hemispherical applicator 3A as well as the elongated, axisymetric, cylindrical applicator 3B which rotates about an elongate axis 358 to roll over the upper surface of the nail plate.

It is recognized that it may be desired to protect the upper surface of adjacent tissue 354 (i.e. not covered by a nail plate) from accidental contact with the applicator 3 which may burn the patient. Thus, in exemplary embodiments, a protective cover (for example, saran wrap) is placed on the upper surface of adjacent tissue 354.

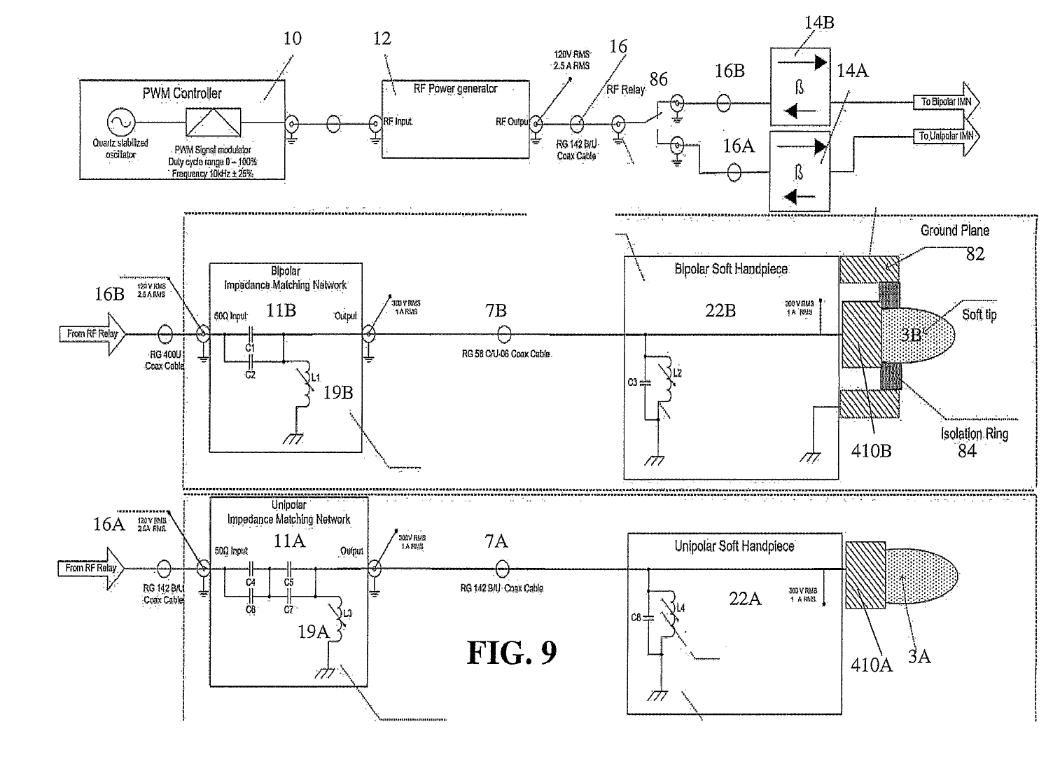

FIG. 9 illustrates a schematic diagram of an exemplary device configured to operate either as a unipolar device (i.e. with a unipolar handpiece 22A) or as a bipolar device (i.e. with bipolar handpiece 22B). According to the example of FIG. 9, the device provides a switch 86 which may direct an electrical signal to either (a) a bipolar array including a bipolar impedance matching network 11B and a bipolar handpiece 22B or (b) a unipolar array including a unipolar impedance matching network 11A and a unipolar handpiece 22A. The bipolar handpiece 22B includes a ground plane 82 which functions as a “return electrode” and is electrically isolated from the applicator 3B by an isolation ring 84. The unipolar handpiece 22A lacks the return electrode and provides a single electrode, namely applicator or coupler 3A.

The example of FIG. 9 depicts a device that provides both bipolar as well as unipolar treatment. It is appreciate, however, that exclusively bipolar devices as well as exclusively unipolar devices are within the scope of the present invention.

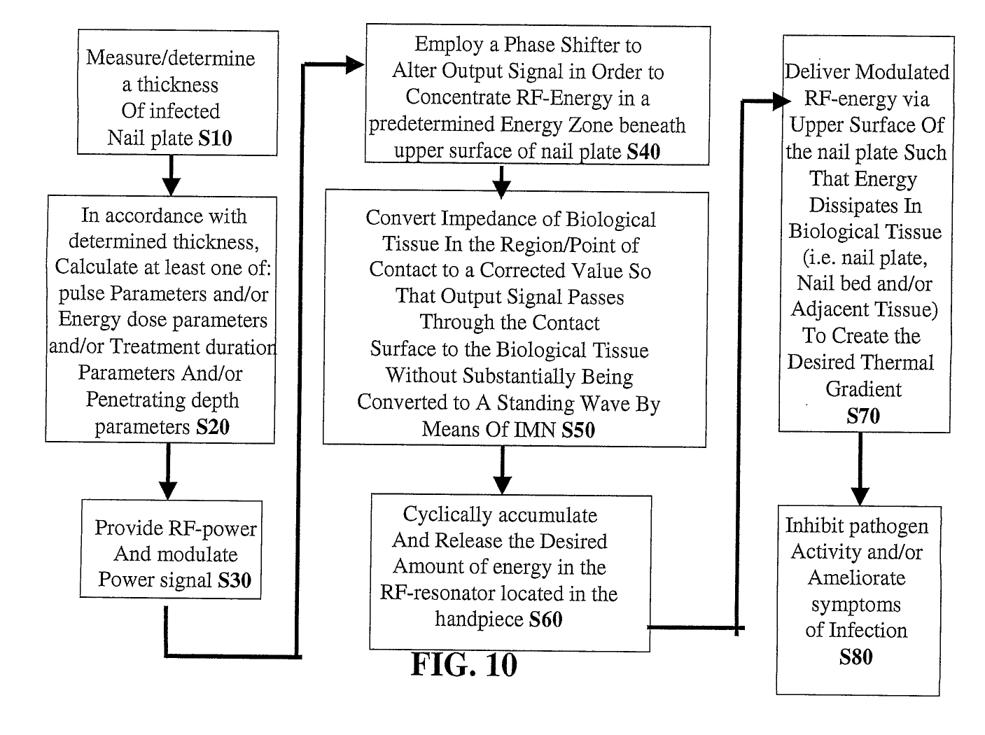

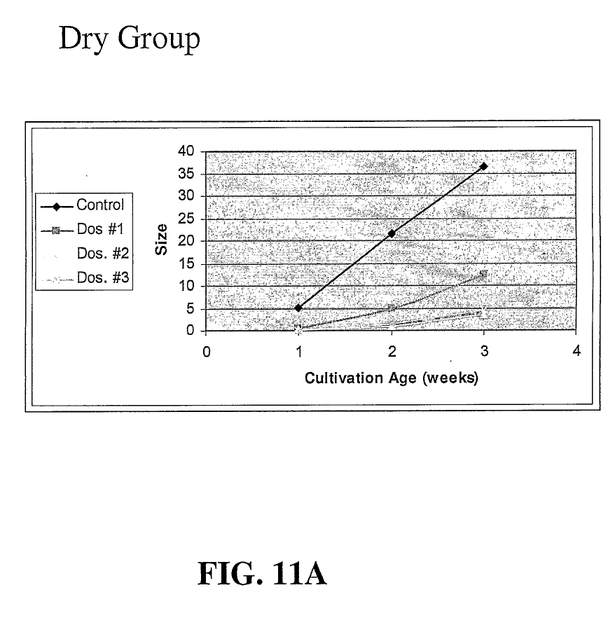

FIG. 10 illustrates a flow-chart of an exemplary method for treating nail conditions and/or for operating a device in accordance with exemplary embodiments of the present invention. In some embodiments, the thickness of the nail is determined S10, and pulse parameters and/or energy does parameters and/or treatment duration parameters and/or penetrating depth parameters are then determined or calculated S20 in accordance with the nail thickness. For example, if it is determined that the nail is thicker, an increased energy level or treatment duration may be selected. In one example, the energy dose E (i.e. the total amount of RF and/or microwave radiation delivered in units of joules) may be calculated as E=kh, where h is the thickness of the nail plate, and k is an empirical coefficient dependent one or more of nail plate irregularity, operating frequency, phase shift and method of energy deposition in particular the ratio between amplitude and duty cycle of RF and/or microwave-pulses. The measurement may be performed “automatically” (for example, by providing a device that also includes a mechanism operative to measure nail thickness, for example, a laser device for measuring nail thickness) or manually.

It is noted that in some embodiments, parameters may be selected without first measuring the thickness of the nail plate.

An electromagnetic (e.g. RF and/or HF and/or microwave) power signal is provided and modulated S30 in accordance with pulse parameters. A phase shifter 14 is employed S40 to alter the output power signal to concentrate electromagnetic (e.g. RF and/or HF and/or microwave) energy in certain energy zone—for example, the nail plate and/or the upper region of the nail bed. This may be useful so that the delivered energy does not penetrate too deeply below the surface of the nail bed which could burn the patient and/or inflict too great a pain. The impedance of biological tissue (for example, nail plate) in the region and/or point of contact is converted S50 so that the output signal passes through the surface of the biological tissue (for example, the upper surface of the nail plate) without substantially being converted to a standing wave by means of an IMN 11. Energy is cyclically accumulated and released S60 in the resonator located in the handpiece 22.

Modulated electromagnetic (e.g. HF and/or RF and/or microwave) energy is delivered S70 to the biological tissue (i.e. nail plate and/or nail bed and/or nail matrix and/or other adjacent tissue) to create a desired thermal gradient S70 featuring a temperature high enough to inhibit pathogen activity S80 while below the energy delivery zone, the temperature remains low enough so as not to inflict no or minimal undesired pain and/or minimal or no tissue damage. Pathogen activity may be inhibited for example, by killing or damaging pathogens with the high temperature and/or reducing the activity of existing pathogens.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### A Model System

Experiments to illustrate the effectiveness of RF radiation in reducing the population of fungal pathogens were conducted using an in-vitro model system. According to the model system, fungal mass and spores were taken from fungus-infected toenails and placed beneath an “upper layer” of glass (i.e. thickness 1 mm) which represented the nail plate, and placed on a half-apple (i.e. with the plane or flat region of the half-apple oriented upwards) which represented the finger (i.e. had a similar impedance to biological tissue of the patient). This model system was constructed a number of times, and for each model system, the “fungal infection” was subjected to a specific respective “treatment.” In order to biologically isolate the fungus from the apple, a thin layer of glass (i.e. thickness 0.3 mm) was placed below the fungus, and separated between the fungus and the apple surface.

The model system was constructed as follows: fungus samples were harvested from fungally-infected toenails of several patients, and were placed in between two layers of glass—the first “thicker” layer having a thickness of 1 mm, and a second “thinner” layer having a thickness of 0.3 mm. Each structure including the fungus samples between the two layers of glass was placed on a respective half-apple, oriented so that the thicker end was up.

A total of 80 model systems were constructed, and were divided into 8 groups. Four groups were designated as “wet groups”—model systems in the wet groups (i.e. where each fungus sample was mixed with urea prior to treatment in order to increase the water content in the fungus' environment). Four groups were designated as “dry groups” where no urea was applied.

There were two control groups that were not subjected to RF radiation treatment—a “dry” control group of 10 model systems and a “wet” control group of 8 model systems. The six remaining groups were given different dosages of RF treatment. RF treatment was applied using a unipolar device having an RF power source at 40.68 MHz, spherical conformable conducting applicator (coupler (i.e. rubber with embedded metal particles), an IMN, a pulse-width modulator, resonator, and a phase shifter.

Different groups were administered different doses. Two groups (i.e. one “dry group” of 9 model systems and one “wet group” of 12 model systems) were administered a first dosage (“dosage #1”) of 480 W, for 6 seconds. Two groups (i.e. one “dry group” of 10 model systems and one “wet group” of 10 model systems) were administered a second dosage (“dosage #2”) of 600 W, for 4 seconds. Two groups (i.e. one “dry group” of 10 model systems and one “wet group” of 11 model systems) were administered a first dosage (“dosage #3”) of 600 W, for 6 seconds. After the radiation was administered, for each model system, the respective fungus sample was harvested and separately cultured in a respective Petri dish.

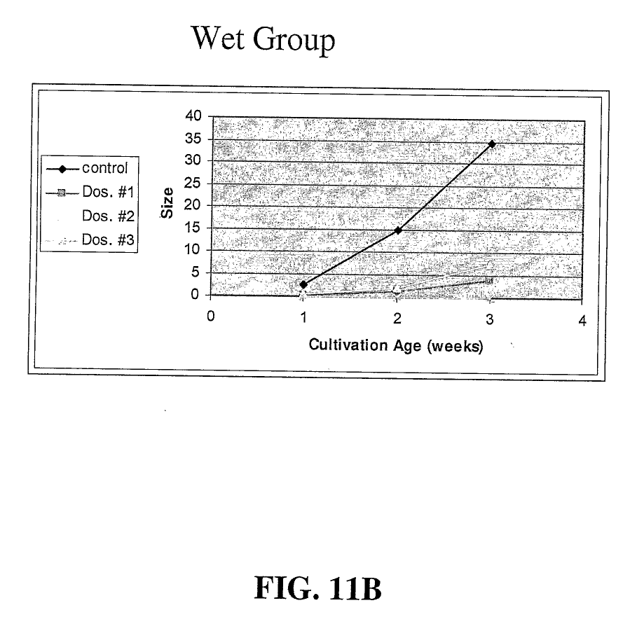

The size of the fungus cultures were measured for each respective dish after 1, 2 and 3 weeks. The sizes for the different groups are reported in FIGS. 11A-11B as a function of time.

Results are also reported in Tables 1 and 2.

TABLE 1
DRY GROUP
P values of differences between culture sizes

| | 1 week | 2 week | 3 week |
|---|---|---|---|
| Control vs Dos. #1 | 0.1374 | 0.0428 | 0.0354 |
| Control vs Dos. #2 | 0.1337 | 0.0141 | |
| Contorl vs Dos. #3 | 0.1180 | 0.0428 | 0.0036 |

TABLE 2
WET GROUP
P values of differences between culture sizes

| | 1 week | 2 week | 3 week |
|---|---|---|---|
| Control vs Dos. #1 | 0.0087 | 0.0107 | 0.0004 |
| Control vs Dos. #2 | 0.0087 | 0.0094 | |
| Contorl vs Dos. #3 | 0.0056 | 0.0071 | |

In the tables, the lower P value indicates a more statistically significance difference between a particular group and the control.
All 3 dosages of RF irradiation inhibited fungus growth in both groups. The most statistically significant difference between the treated group and three control groups was observed after 3 weeks in dosage 2 of the dry group, and after 3 weeks in dosages 1-3 of the wet group.

It is concluded that it is possible to reduce a nail fungus population with RF radiation, and in many situations, treatment with urea increases the effectiveness of fungus population reduction up to complete eradication of fungus mass.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

The present inventors are aware that the presently disclosed conducting, conformable applicator may be used to delivery high-frequency electromagnetic energy to hard tissue (including but not limited to non-mineralized hard tissue) other than nail plates, and thus, various teachings of the present invention may apply to other hard tissues.

The present inventors are aware that certain teachings of the present invention may be used to infectious conditions and/or inflammatory conditions (including but not limited to psoriasis) other than fungal nail infection, and to reduce the activity and/or the population and/or kill pathogens (either active and/or spores) other than fungal pathogens.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

All references cited herein are incorporated by reference in their entirety. Citation of a reference does not constitute an admission that the reference is prior art.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

1) Apparatus for treating a human nail of a subject, the apparatus comprising:
a) an electromagnetic energy source operative to produce electromagnetic energy having a frequency of at least 0.5 MHZ (megahertz) and less than 10 GHZ (gigahertz), said electromagnetic energy source configured to produce an output electromagnetic signal directed to a conductive conformable applicator contactable with an upper surface of the nail of the subject;
b) a pulse controller operative to cause said electromagnetic energy source to deliver said output electromagnetic signal as a pulsed signal having one or more pre-determined pulse parameters, said pulse controller operative to effect a pulse-width modulation of said pulsed signal;
c) an impedance transformer operative to convert an impedance of tissue associated with the nail so that traveling waves of said pulsed output signal reach the human nail substantially without being converted to a standing wave; and
d) said conductive conformable applicator for delivering said electromagnetic pulsed output signal to the nail, said conformable applicator configured to conform to a shape of said upper surface of the nail plate upon contact with the nail,
wherein said apparatus is operative such that said delivery of said pulsed signal is effective to heat at least one of a nail plate tissue and nail bed tissue below said nail plate.

2) Apparatus of claim 1 wherein said electromagnetic energy source is selected from the group consisting of an HF energy source, an RF energy source and a microwave energy source.

3) Apparatus of claim 1 wherein the apparatus is operative to deliver said pulse signal to heat at least one of a nail plate tissue and nail bed tissue below said nail plate to a temperature sufficient to inhibit activity of a pathogen residing within said heated tissue.

4) Apparatus of claim 1 wherein the apparatus is operative to deliver said pulse signal to heat at least one of a nail plate tissue and nail bed tissue below said nail plate to a temperature sufficient to inhibit activity of a fungal pathogen residing within said heated tissue.

5) Apparatus of claim 1 wherein the apparatus is operative to deliver said pulse signal to heat at least one of a nail plate tissue and nail bed tissue below said nail plate to at least 50 degrees Celsius

6) Apparatus of claim 1 wherein the apparatus is operative such that said heating produces a sharp temperature gradient having a temperature variation of at least 20 degrees/millimeter over a distance of at least 0.1 mm in at a location below said surface of the nail plate

7) Apparatus of claim 6 wherein said location of said temperature gradient is within the nail plate.

8) Apparatus of claim 6 wherein said location is within 1 mm of a nail plate-nail bed interface.

9) Apparatus of claim 6 wherein said location is within a given distance from the nail plate-nail bed that is at most the thickness of the nail plate.

10) Apparatus of claim 6 wherein the apparatus is operative to produce said sharp temperature gradient in accordance with said one or more pre-determined pulse parameters of one or more pulses delivered from said energy source.

11) Apparatus of claim 1 wherein at least one said pulse parameter is selected from the group consisting of an amplitude, pulse duration, a pulse shape, a duty cycle parameter, a pulse sequence parameter, a pulse rise-time, and a pulse frequency.

12) Apparatus of claim 11 wherein said pulse generator is operative to establish a value of said duty cycle parameter that is between 1% and 100%.

13) Apparatus of claim 11 wherein said pulse generator is operative to establish a value of said duty cycle parameter that is between 15% and 30%.

14) Apparatus of claim 11 wherein said pulse generator is operative to establish a value of said pulse duration between 10 nanoseconds and 10 milliseconds.

15) Apparatus of claim 14 wherein said pulse duration is between 1 and 100 microseconds.

16) Apparatus of claim 15 wherein said pulse generator is operative to establish a rectangular pulse shape.

17) Apparatus of claim 1 further comprising
e) a phase shifter operative to control a phase of said traveling waves of said pulsed output signals so that a location of maximum amplitude is substantially at said upper surface of said nail plate within a tolerance that is at most 0.5 a thickness of said nail plate.

18) Apparatus of claim 17 wherein said tolerance is at most 0.2.

19) Apparatus of claim 1 wherein said conformable conductive applicator includes:
a) a first non-conducting conformable material; and
b) a plurality of particles of a second conducting material, said particles embedded within said first material.

20) Apparatus of claim 19 wherein said first non-conducting material is an electrical insulator.

21-96. (canceled)
